(19) United States
(12) Patent Application Publication
(10) Pub. No.: US 2018/0024109 A1
(43) Pub. Date: Jan. 25, 2018

(54) Tissue Culture Tray and System for the In Vitro Testing and Characterization of Magnetically-Induced Rotation and Translational Motion of Magnetic Particles

(71) Applicant: The Board of Trustees of The University of Illinois, Urbana, IL (US)

(72) Inventor: Herbert H. Engelhard, III, Wilmette, IL (US)

(21) Appl. No.: 15/655,471

(22) Filed: Jul. 20, 2017

Related U.S. Application Data
(60) Provisional application No. 62/365,606, filed on Jul. 22, 2016.

(51) Int. Cl. G01N 33/483; C12M 1/42; G01R 33/12; C12M 1/12; C12M 1/34; C12M 3/00; C12M 1/32

(52) U.S. Cl. CPC G01N 33/4833; C12M 21/08; C12M 35/06; C12M 23/12; C12M 23/06; C12M 41/12; G01R 33/1276

(57) ABSTRACT

A tissue culture plate or tray and a system for magnetically induced rotational and translational (“MIRT”) movement of magnetic particles are disclosed. A representative plate or tray comprises: a plate base having an upper surface and a plurality of sides, the plate base having a form factor of a truncated cuboid; and a plurality of channels arranged in the plate base, each channel spaced apart from one or more sides of the plurality of sides, each channel having a predetermined depth from the upper surface of the plate base, and each channel having a length and a width, with the length being greater than the width. The channels may be parallel, or branched, or curved or straight, and may have variable widths. The channels may be tubular or open to the upper surface of the plate base. A MIRT system using the tissue culture plate or tray is also disclosed.

### CROSS-REFERENCE TO A RELATED APPLICATION

This application is a nonprovisional of and claims the benefit of and priority to U.S. Provisional Patent Application No. 62/365,606, filed Jul. 22, 2016, inventor Herbert Engelhard, titled “Tissue Culture Plate for the Study of Magnetically Induced Rotary Traction”, which is commonly assigned herewith, and all of which is hereby incorporated herein by reference in its entirety with the same full force and effect as if set forth in its entirety herein.

### FIELD OF THE INVENTION

The present invention, in general, relates to a system that allows testing of translational motions of magnetic particles, and in particular, relates to an in vitro system and tissue culture tray which sufficiently or generally may simulate relevant biological conditions of conduits within the human body or other animal, such as vasculature and neurological spaces, thereby providing capability for in vitro testing and characterization of translational motion of magnetic particles, especially by magnetically-induced rotation and translation.

### BACKGROUND OF THE INVENTION

Many investigators, especially those involved in designing therapeutic particles, are currently designing and studying magnetic nanoparticles and/or aggregates of magnetic particles for use against many types of diseases. Recently, there has been enthusiasm for using magnetic nanoparticles (“MNPs”) as theranostic agents, particularly for cancer (Kang PMID: 26528835, Nandwana 26102385).

Nanoparticles are useful in imaging and drug delivery. The use of magnetic guidance for medical purposes, including use against cancer and in the central nervous system (CNS), has long been an attractive idea. There have been concerns, however, about potential MNP toxicity, non-specific binding, and/or applicability to human-sized distances. To date, such issues have been addressed in vitro and in animal models only on a limited basis, primarily with the use of static (i.e. nonrotating) permanent magnets.

MNPs as theranostic agents, particularly for cancer, including superparamagnetic iron oxide nanoparticles (“SPIONs”), can be readily produced, functionalized, made fluorescent, and can be visualized by magnetic resonance imaging (“MRI”), for example and without limitation (e.g., Singh 24910878). Particles that are SPIONs in isolation actually may have strong magnetic dipole interactions, and form a collective state or aggregate which is temperature-dependent (Morop 21977409; Wilson 20161001).

MNPs have been extensively described and studied. The design of nanoparticle size, shape and surface chemistry certainly plays an important role in processes that are critical for therapeutic delivery such as biodistribution, cell internalization and subcellular localization (Liu 22834196; Haun 18630976, Cicha 26468004). Particle uptake by cultured cells can be affected by factors including serum in the growth medium, and the type of drug or other molecule that is bound to the MNP (Cicha 26468004). Studies comparing cancer to non-neoplastic cells are lacking, and finding suitable controls is difficult. While theoretical models have been described (Carrillo 23231258), these need to be tested in suitable preclinical models. Several approaches have been used to model nanoparticle magnetization dynamics (Reeves 25271360). While a wide variety of designs are possible, MNPs most commonly are comprised of an iron oxide core, which is coated with gold or polyethylene glycol (PEG) and functionalized with the therapeutic drug (or other molecule) of choice. MNPs can readily be made fluorescent, such as by using quantum dots (QD) for example and without limitation, and a variety of other methods have been described concerning how they can be further modified, including for detection and imaging. Doxorubicin (DOX) has often been a drug that has been conveyed using MNPs (Rudge 10872770).

Many of these studies take place in standard, prior art well culture plates or Petri dishes. In many cases, however, it would be highly desirous to study the movement and uptake of magnetic particles (such as MNPs) along tubes or conduits in the body of an animal, naturally occurring or implanted, such as human blood or lymph vessels or an implanted cardiac stent. The well culture plates and Petri dishes currently in use for this application do not allow researchers to adequately study or model linear or curvilinear movement of such magnetic particles. For example, as shown in FIG. 26, MNPs 75 placed in a well culture plate or Petri dish 50, with a starting location 70 and then subject to the application of magnetic fields, simply spread out in multiple directions in this environment, resulting in a larger circle (or spot) 80, which does not adequately allow for the detection, study, characterization and modeling of the linear or curvilinear movement of such magnetic particles, especially with respect to their behavior in structures such as blood vessels. An alternative would be to study the in vivo movement and uptake of magnetic particles in animals, but this research is costly and time-consuming, and also may not adequately mimic human-sized distances, which is very important when employing magnetic targeting.

Accordingly, a need remains for a versatile tissue culture plate, tray or dish, and a system, for in vitro testing and characterization of the translational motion of magnetic particles such as MNPs, especially by magnetically-induced rotation and translation. Such tissue culture plates, dishes or trays should also provide for the capability to at least partially model the translational motion of magnetic particles in human or other animal vasculature and other conduits, whether natural or implanted, in the human body or other animal.

### SUMMARY OF THE INVENTION

The representative embodiments of the present invention provide numerous advantages. An in vitro system is disclosed which generally can be made to more closely simulate conditions in conduits within the human body, such as vasculature, tubing, stents, or shunts, and the actual human-sized distances that would exist during treatment. The various representative tissue culture plate or tray embodiments provide the capability for researchers to study and model in vitro the linear or curvilinear movement (e.g., velocity) and uptake of magnetic particles along channels, and to control significant parameters, including types of cells lining the channels, and the type and viscosity of any media, such as plasma or other fluid in the channel. The disclosed representative tissue culture plate or tray embodiments and system provide the foundation for the study and testing of the linear or curvilinear translational movement of magnetic particles (and/or drug targeting), especially by magnetically-induced rotation and translation (“MIRT”), prior to any animal studies or clinical trials. As a further advantage, the various representative embodiments allows researchers to study the use of magnetic particles for treatment of diseases such as stroke, cancer, and coronary artery disease, for example and without limitation. In particular, it may allow studies involving the design and testing of magnetic particles, especially nanoparticles such as magnetic nanoparticles (MNPs) or superparamagnetic iron oxide nanoparticles (SPIONs), for example.

It should be noted that while referred to as a tissue culture plate or tray, the representative embodiments are not so limited and may be utilized for any other purposes, including those which do not involve tissue cultures. For example, the representative tissue culture plate or tray embodiments may be utilized without cell cultures or other cellular involvement, such as to model the movement of magnetic particles in conduits such as shunts and catheters, for example and without limitation.

There are numerous examples of the types of uses of the representative tissue culture plate or tray and system embodiments. For example, blood clots can be formed in the channels of the representative tissue culture plate or tray embodiments, as described in greater detail below. Different doses of magnetic particles (having drugs or other therapeutic molecules) may then be placed into the starting positions of the channels of the representative tissue culture plate or tray embodiments, followed by transporting the magnetic particles magnetically to the clots, enabling the study of the dissolution of the clots. Not only clots, but tissue (such as brain slice), or membranes, or porous material simulating tissue, may be placed in the channels 110, 210, for example, to test what particle size (e.g., 20 nm) may be required to use the system 700 to move the magnetic particles 70 through that tissue. Also for example, one or more parts of the channels of the representative tissue culture plate or tray embodiments may be cultured with tumor cells, and the representative tissue culture plate or tray embodiments may be utilized to study and model the delivery of chemotherapeutic agents using magnetic particles, such as through artificial cerebrospinal fluid bathing the cultured tumor cells.

Also significant, the representative tissue culture plate or tray embodiments can be fabricated to be compatible with virtually any kind or type of imaging technique, such as light microscopy, photography, video recording, fluorescent microscopy, MM scan, ultrasound scan, CT scan and other x-ray imaging or other spectroscopy, for example and without limitation. In addition, as discussed in greater detail below, different types of monitors may be utilized in the channels of the representative tissue culture plate or tray embodiments, such as fluorescence or magnetic detectors, also for example and without limitation.

Representative embodiments of a tissue culture plate or tray are disclosed for magnetically induced rotational and translational movement of magnetic particles, with a representative the tissue culture plate or tray embodiment comprising: a plate base having an upper surface and a plurality of sides, the plate base having a form factor of a truncated cuboid; and a plurality of channels arranged in the plate base, each channel of the plurality of channels spaced apart from one or more sides of the plurality of sides, each channel of the plurality of channels having a predetermined depth from the upper surface of the plate base, and each channel of the plurality of channels further having a length and a width, wherein the length is greater than the width. For example, the truncated cuboid defines a hexagon in the lateral dimension.

In a representative embodiment, the plurality of channels may be arranged to be either parallel, or as a plurality of branches of a central channel of the plurality of channels. For example, the plurality of branches may be curved branches, or substantially straight branches, or a combination of curved and substantially straight branches.

In a representative embodiment, the plurality of channels conform to a human or animal morphology.

Also in a representative embodiment, the plurality of channels may be arranged as a plurality of straight or curved branches or both straight and curved branches of a central channel of the plurality of channels, with the central channel having a first width or diameter and the plurality of branching channels having second or more widths or diameters which are successively or incrementally smaller than the first width or diameter.

In a representative embodiment, at least one channel of the plurality of channels has a smooth surface texture. In another representative embodiment, at least one channel of the plurality of channels has a non-smooth surface texture.

In a representative embodiment, at least one channel of the plurality of channels has a varying width or depth.

In a representative embodiment, at least one channel of the plurality of channels has a width from one (1.0) mm to ten (10) mm, such as at least one channel of the plurality of channels having a width from three (3) mm to five (5) mm, or at least one channel of the plurality of channels having a width from one (1.0) mm to one and one-half (1.5) mm.

In another representative embodiment, at least two channels of the plurality of channels are coupled to each other.

In a representative embodiment, the plate base is comprised of a material compatible with an imaging technique selected from the group consisting of magnetic resonance imaging, x-ray imaging, computed tomography (CT) imaging, ultrasound imaging, light microscopy, fluorescence microscopy, and combinations thereof.

In various representative embodiments, the plate base has an overall form factor matching a standard 96-well culture plate, or a standard 96-well culture plate, or a standard 48-well culture plate, or a standard 16-well culture plate, or a standard 6-well culture plate.

In another representative embodiment, the plate base further comprises: a skirt arranged around a periphery of the plate base.

In a representative embodiment, at least one channel of the plurality of channels is at least partially open to the upper surface of the plate base. In another representative embodiment, at least one channel of the plurality of channels is open to the upper surface of the plate base only at a first channel end and at one or more second channel ends, and wherein the region of the at least one channel between the first and second channel ends is closed to the upper surface of the plate base.

In another representative embodiment, at least one channel of the plurality of channels has a semicircular channel bottom. In another representative embodiment, at least one channel of the plurality of channels is open to the upper surface of the plate base and has a bottom surface having a cross-sectional shape selected from the group consisting of: semi-circular, square, rectangular, semi-elliptical, polygonal, triangular, and combinations thereof.

In another representative embodiment, at least one channel of the plurality of channels is tubular. For example, at least one channel of the plurality of channels may be tubular and have a cross-sectional shape selected from the group consisting of: circular, square, rectangular, elliptical, polygonal, triangular, and combinations thereof.

In another representative embodiment, a tissue culture plate or tray for magnetically induced rotational and translational movement of magnetic particles, with the tissue culture plate or tray comprising: a plate base having an upper surface and a plurality of sides, the plate base having a form factor of a truncated cuboid, wherein the plate base is comprised of a material compatible with an imaging technique selected from the group consisting of magnetic resonance imaging, x-ray imaging, computed tomography (CT) imaging, ultrasound imaging, light microscopy, fluorescence microscopy, and combinations thereof and a plurality of channels arranged in the plate base, each channel of the plurality of channels spaced apart from one or more sides of the plurality of sides, each channel of the plurality of channels having a predetermined depth from the upper surface of the plate base, and each channel of the plurality of channels further having a length and a width, wherein the length is greater than the width; wherein the plurality of channels are arranged as a plurality of straight or curved branches of a central channel of the plurality of channels, the central channel having a first width or diameter and the plurality of branching channels having widths or diameters which are successively or incrementally smaller than the first width or diameter.

A representative system embodiment is disclosed for magnetically induced rotary translational movement of magnetic particles in a tissue culture plate or tray, with the system comprising: a magnetic device to generate a rotating magnetic field having a time-varying amplitude and polarity; and a tissue culture plate or tray spaced-apart from the magnetic device a predetermined lateral distance and elevation, the tissue culture plate or tray comprising: a plate base having an upper surface and a plurality of sides, the plate base having a form factor of a truncated cuboid; a plurality of channels arranged in the plate base, each channel of the plurality of channels spaced apart from one or more sides of the plurality of sides, each channel of the plurality of channels having a predetermined depth from the upper surface of the plate base, each channel of the plurality of channels further having a length and a width, wherein the length is greater than the width.

In a representative embodiment, the predetermined lateral distance and elevation is selected from the group consisting of: a pull position laterally offset from the magnet in a first direction; a push position laterally offset from the magnet in a second direction, the second direction opposite the first direction; a centered position having a predetermined elevation above or below the magnet without a lateral offset; an offset position having a predetermined elevation above the magnet with a lateral offset; and combinations thereof.

A representative system may further comprise: an adjustable plate mount removably couplable to the tissue culture plate or tray, the adjustable plate mount configured to position the tissue culture plate or tray at user-selectable, varying distances and elevations from the magnet.

A representative system may further comprise: a heating unit configured to heat contents of the tissue culture plate or tray to a predetermined temperature or to maintain the tissue culture plate or tray at the predetermined temperature.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is an isometric view of a representative first tissue culture plate or tray embodiment.

FIG. 2 is a plan view of the representative first tissue culture plate or tray embodiment.

FIG. 3 is a plan view of a representative second tissue culture plate or tray embodiment.

FIG. 4 is a plan view of a representative third tissue culture plate or tray embodiment.

FIG. 5 is a first cross-sectional view of the representative first, second and/or third tissue culture plate or tray embodiments illustrated in FIGS. 1-4.

FIG. 6 is a second cross-sectional view of the representative first, second and/or third tissue culture plate or tray embodiments illustrated in FIGS. 1-4 showing additional channel configurations or shapes and surface textures.

FIG. 7 is a third cross-sectional view of the representative first, second and/or third tissue culture plate or tray embodiments illustrated in FIGS. 1-4 having various coatings, cell cultures, and media.

FIG. 8 is an isometric view of a representative fourth tissue culture plate or tray embodiment.

FIG. 9 is a plan view of the representative fourth tissue culture plate or tray embodiment.

FIG. 10 is a plan view of a representative fifth tissue culture plate or tray embodiment.

FIG. 11 is a plan view of a representative sixth tissue culture plate or tray embodiment.

FIGS. 12A and 12B are first and second cross-sectional views of the representative fourth tissue culture plate or tray embodiment illustrated in FIG. 9.

FIG. 13 is a third cross-sectional view of the representative fourth, fifth and/or sixth tissue culture plate or tray embodiments illustrated in FIGS. 8-11.

FIG. 14 is a fourth cross-sectional view of the representative fourth, fifth and/or sixth tissue culture plate or tray embodiments illustrated in FIGS. 8-11 showing additional channel configurations or shapes and surface textures.

FIG. 15 is a fifth cross-sectional view of the representative representative fourth, fifth and/or sixth tissue culture plate or tray embodiments illustrated in FIGS. 8-11 having various coatings, cell cultures, and media.

FIG. 16 is an isometric view of a representative first tissue culture plate or tray embodiment having tubes, cylinders and monitors arranged in various channels.

FIG. 17 is a block diagram and elevational view of a representative tissue culture plate or tray system embodiment.

FIG. 18 is a block diagram and elevational view of a representative tissue culture plate or tray system embodiment.

FIG. 19 is a photograph of the representative first tissue culture plate or tray embodiment of FIG. 1 showing linear translational displacement of MNPs following application of a rotating magnetic field.

FIGS. 20A and 20B (collectively FIG. 20) are graphs showing average particle velocity versus distance of a representative tissue culture plate or tray to a rotating magnet in a representative tissue culture plate or tray system embodiment, with FIG. 20A having the representative tissue culture plate or tray embodiment in a centered position, and with FIG. 20B having the representative tissue culture plate or tray embodiment in an offset position, and with the results of FIG. 20B plotted as a function of lateral distance.

FIG. 21 is a graph showing particle velocity versus distance of a representative tissue culture plate or tray, having a centered position, to the center of a rotating magnet (indicated as the origin position) in a representative tissue culture plate or tray system embodiment.

FIG. 22 is a graph showing particle velocity versus distance of a representative tissue culture plate or tray, having a pull position, to a rotating magnet in a representative tissue culture plate or tray system embodiment.

FIG. 23 is a graph showing particle velocity versus distance of a representative tissue culture plate or tray, having a push position, to a rotating magnet in a representative tissue culture plate or tray system embodiment.

FIG. 24 is a graph showing particle velocity versus distance of a representative tissue culture plate or tray, having an offset position, to a rotating magnet in a representative tissue culture plate or tray system embodiment.

FIG. 25 is a graph showing particle velocity versus distance of a representative tissue culture plate or tray, having an offset position, with and without cells, to a rotating magnet in a representative tissue culture plate or tray system embodiment.

FIG. 26 is a photograph of a prior art culture plate or tray showing multidirectional displacement of MNPs following application of a rotating magnetic field.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways (e.g, varying the any of the shapes, configurations and dimensions of the various representative tissue culture plate or tray 100, 200, 300, 400, 500, 600 embodiments and/or channels 110, 210). Also, it is to be understood that the phraseology and terminology employed herein, as well as the abstract included below, are for the purposes of description and should not be regarded as limiting.

As mentioned above, the various representative tissue culture plate or tray 100, 200, 300, 400, 500, 600 embodiments described herein provide the capability for researchers to study and model the linear or curvilinear movement and uptake of magnetic particles along channels 110, 210 (or tubes) in vitro, and to control significant parameters such as cell 150 types and type and viscosity of the media 160. As a result, an in vitro system 700 is provided which sufficiently or generally simulates conditions in conduits within the human body, such as vasculature, tubing, stents, or shunts, which thereby provides the foundation for the study and testing of the linear or curvilinear translational movement of magnetic particles, especially by magnetically-induced rotation and translation (MIRT), prior to any animal studies or clinical trials. As a further advantage, the various representative tissue culture plate or tray 100, 200, 300, 400, 500, 600 and system 700 embodiments allows researchers to study the use of magnetic particles for treatment of diseases such as stroke, cancer, ductal stones, catheter occlusions, and coronary artery disease, with or without cells, and with or without sterile conditions, for example and without limitation. In particular, it may allow studies involving the design and testing of magnetic particles, especially nanoparticles such as magnetic nanoparticles (“MNPs”) or superparamagnetic iron oxide nanoparticles (“SPIONs”, as a subcategory of MNPs), any and all of which are included and referred to herein generally as magnetic particles.

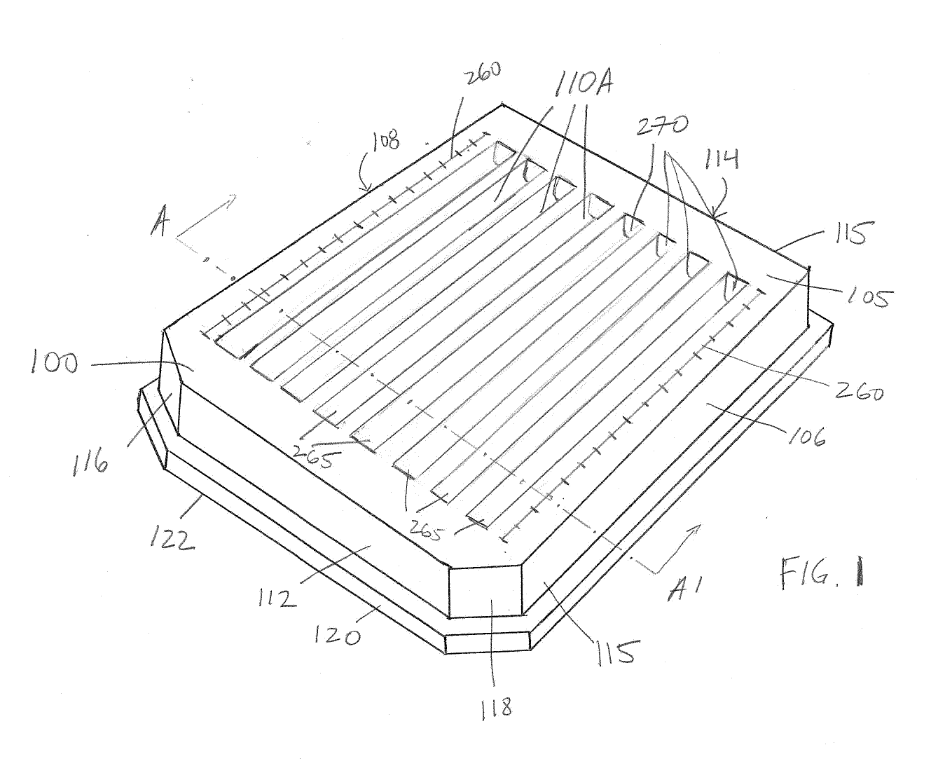

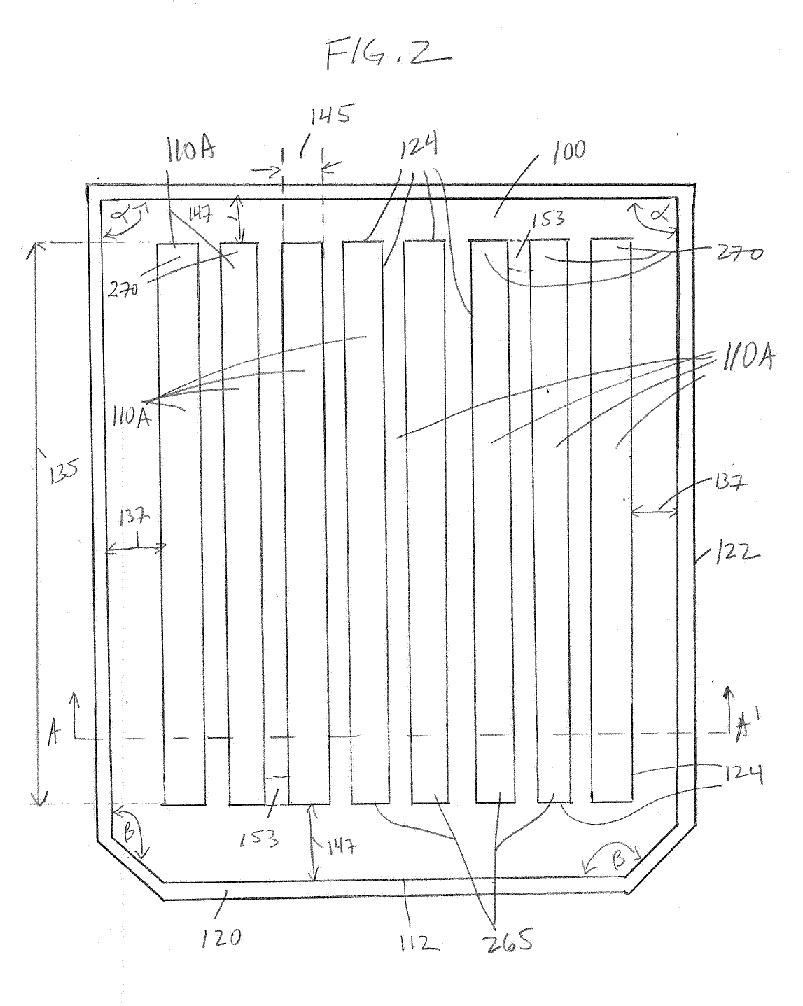

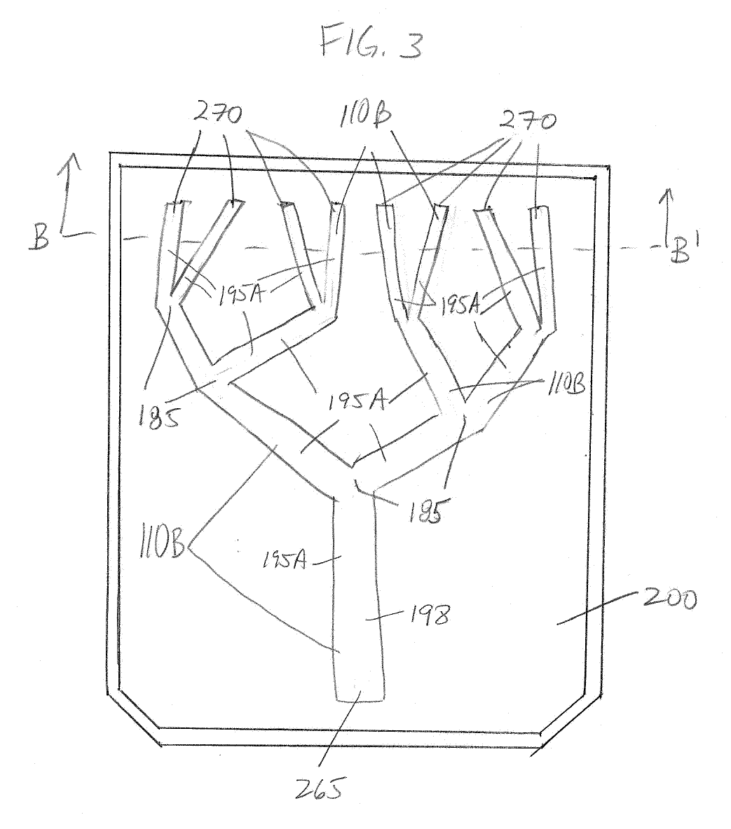

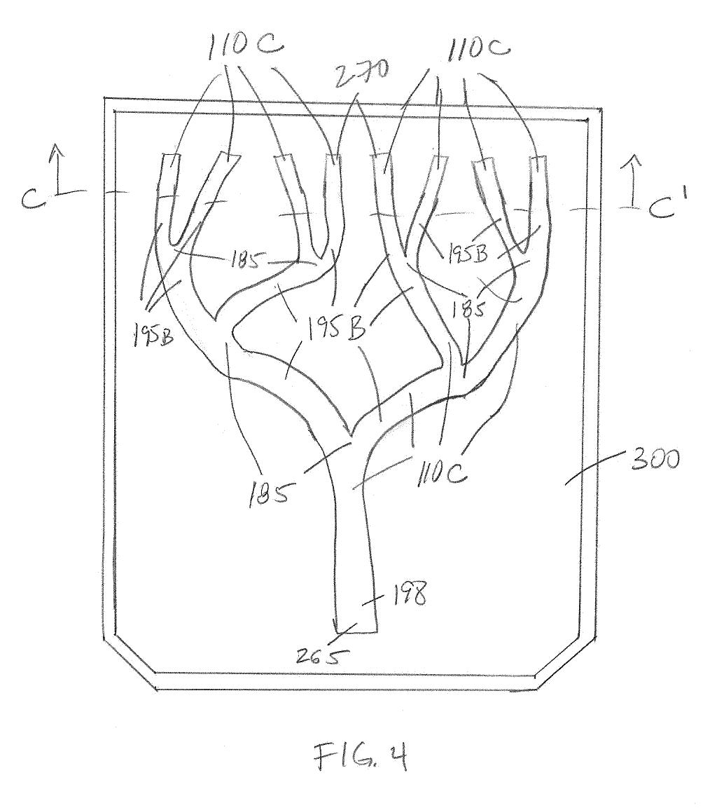

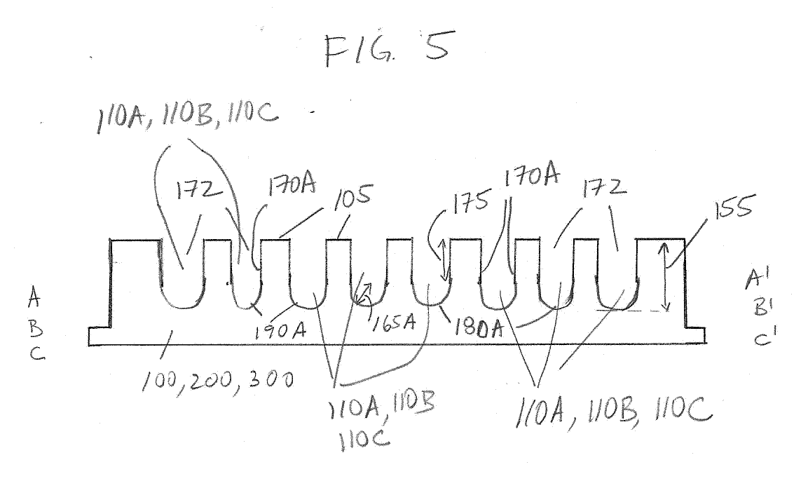

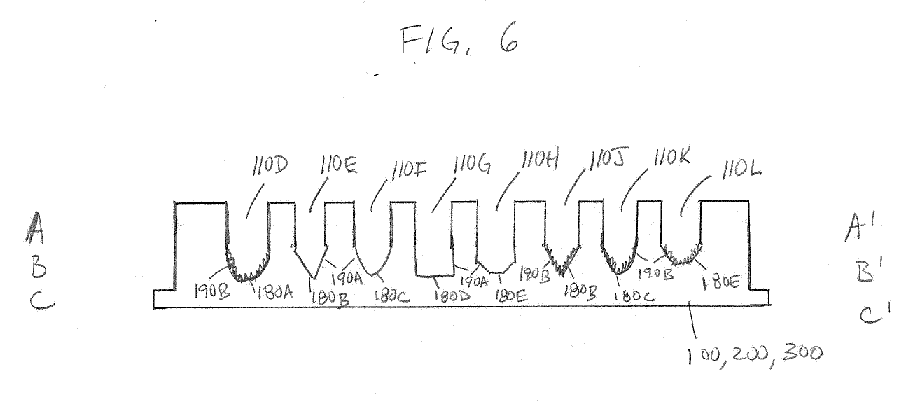

FIG. 1 is an isometric view of a representative first tissue culture plate or tray 100 embodiment. FIG. 2 is a plan view of the representative first tissue culture plate or tray 100 embodiment. FIG. 3 is a plan view of a representative second tissue culture plate or tray 200 embodiment. FIG. 4 is a plan view of a representative third tissue culture plate or tray 300 embodiment. FIG. 5 is a first cross-sectional view of the representative first, second and/or third tissue culture plate or tray 100, 200, 300 embodiments illustrated in FIGS. 1-4. FIG. 6 is a second cross-sectional view of the representative first, second and/or third tissue culture plate or tray 100, 200, 300 embodiments illustrated in FIGS. 1-4 showing additional channel 110 configurations or shapes and surface textures. FIG. 7 is a third cross-sectional view of the representative first, second and/or third tissue culture plate or tray 100, 200, 300 embodiments illustrated in FIGS. 1-4 having various coatings 140, cell cultures 150, and media 160 (such as one or more liquids).

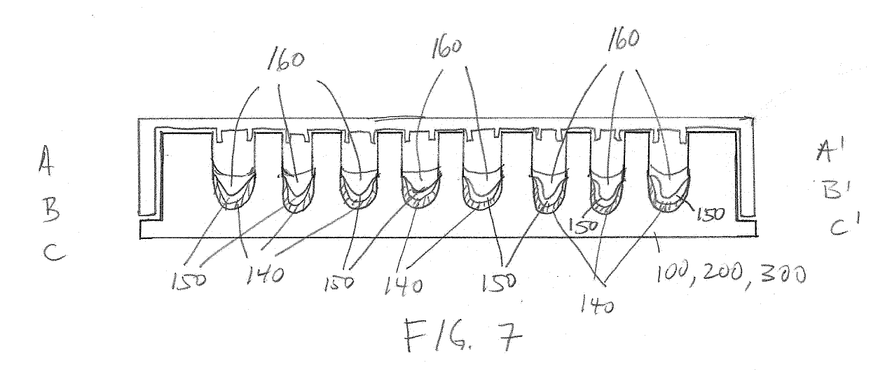

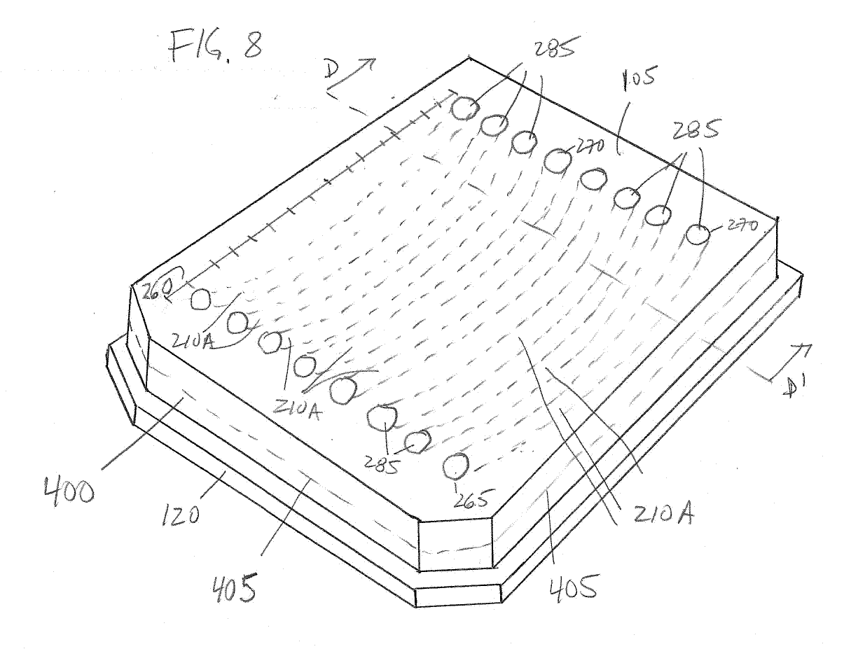

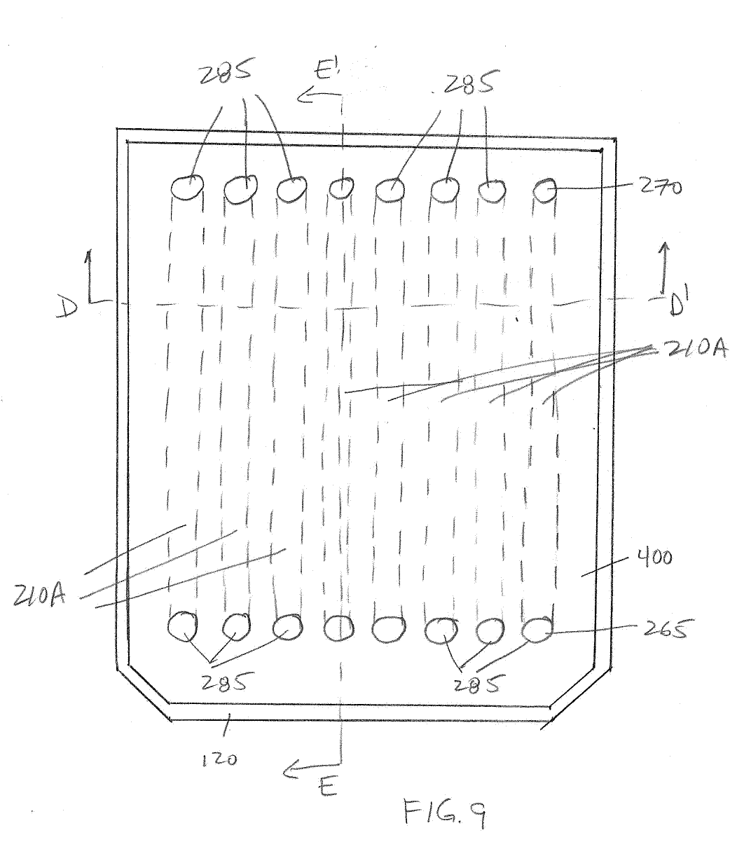

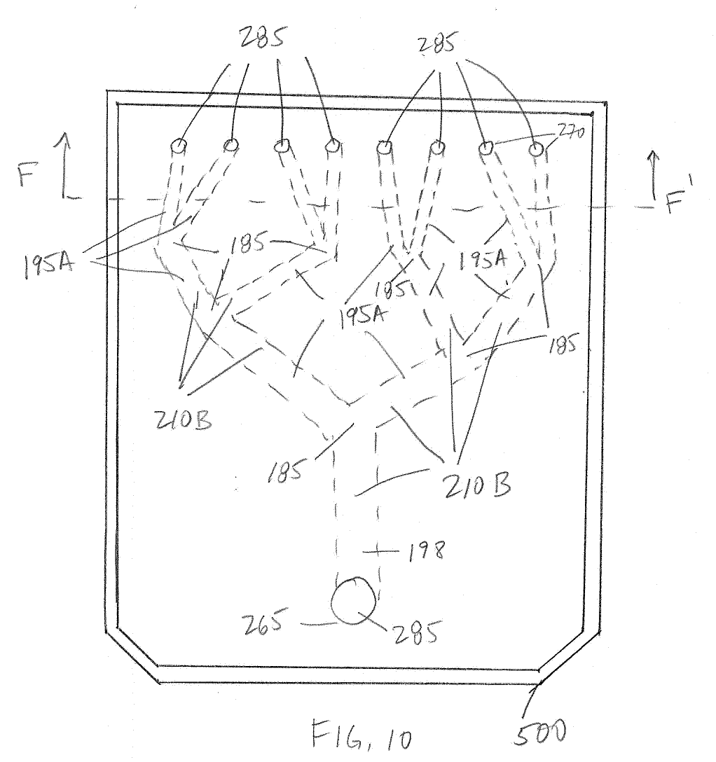

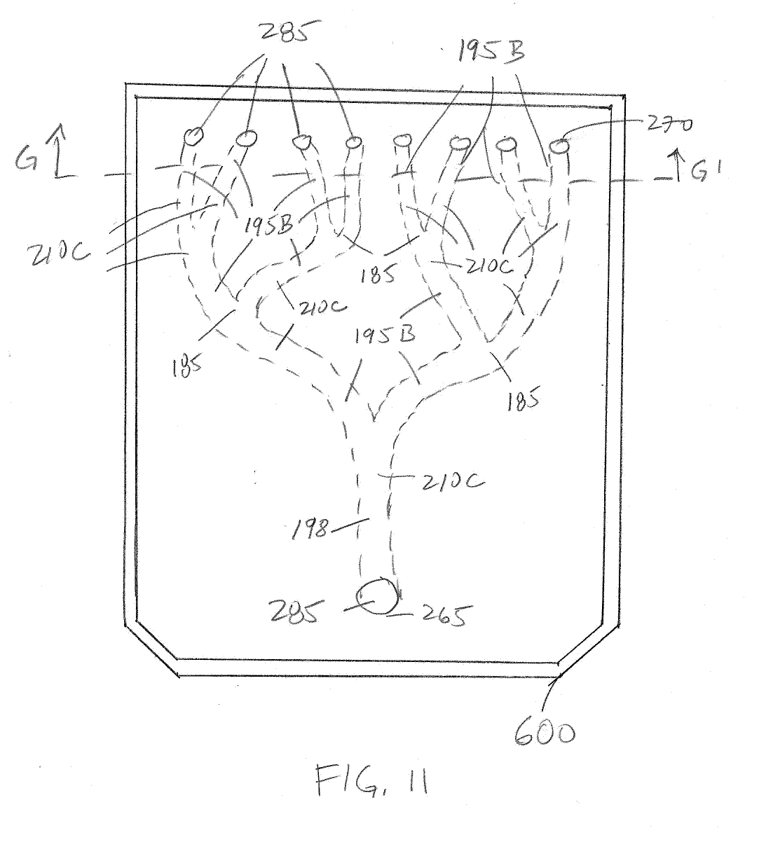

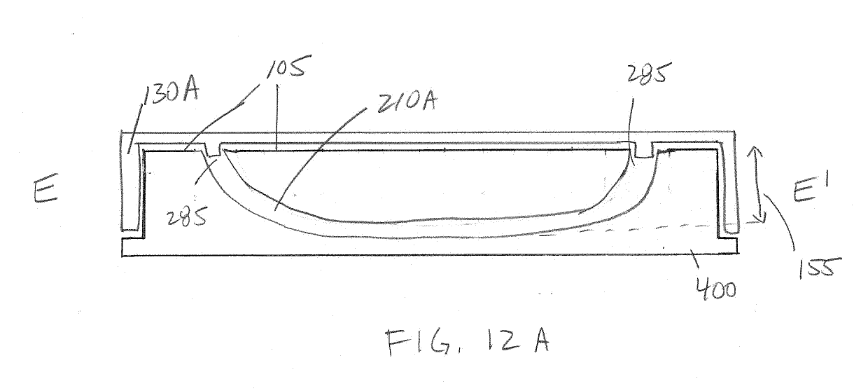

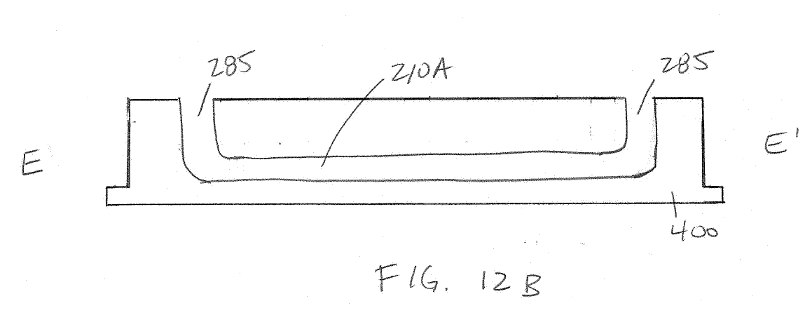

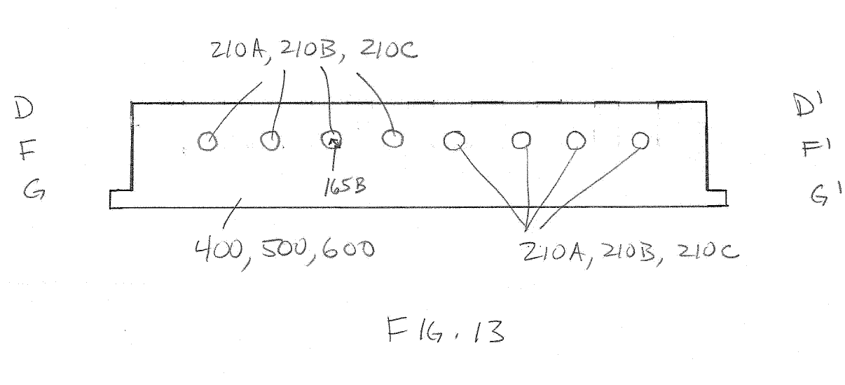

FIG. 8 is an isometric view of a representative fourth tissue culture plate or tray 400 embodiment. FIG. 9 is a plan view of the representative fourth tissue culture plate or tray 400 embodiment. FIG. 10 is a plan view of a representative fifth tissue culture plate or tray 500 embodiment. FIG. 11 is a plan view of a representative sixth tissue culture plate or tray 600 embodiment. The dashed lines in FIGS. 8-11 indicate the extension of the channels 210 below the upper surface 105 of the representative fourth, fifth and/or sixth tissue culture plate or tray 400, 500, 600 embodiments. FIGS. 12A and 12B are first and second cross-sectional views of the representative fourth tissue culture plate or tray 400 embodiment illustrated in FIG. 9, showing example variations on the depth 155 (e.g., variable depths) or extension of the channels 210 below the upper surface 105 of the representative embodiments, such as directly curving below the upper surface (FIG. 12A) or initially extending straight down (perpendicular to the upper surface 105 prior to curving and extending longitudinally and/or laterally), with any and all such variations considered equivalent and within the scope of the disclosure. FIG. 12A also illustrates a lid 130A covering openings 285 at the first and second ends 265, 270. FIG. 13 is a third cross-sectional view of the representative fourth, fifth and/or sixth tissue culture plate or tray 400, 500, 600 embodiments illustrated in FIGS. 8-11. FIG. 14 is a fourth cross-sectional view of the representative fourth, fifth and/or sixth tissue culture plate or tray 400, 500, 600 embodiments illustrated in FIGS. 8-11 showing additional channel configurations or shapes and surface textures. FIG. 15 is a fifth cross-sectional view of the representative representative fourth, fifth and/or sixth tissue culture plate or tray embodiments illustrated in FIGS. 8-11 having various coatings, cell cultures, and media. FIG. 16 is an isometric view of a representative first tissue culture plate or tray 100 embodiment having tubes 340, cylinders 355 and monitors 345 arranged in various channels 110A.

As shown in FIGS. 1-16, the various representative tissue culture plate or tray 100, 200, 300, 400, 500, 600 embodiments may have any overall shape and size, and may be designed to allow use in existing equipment such as standard plate readers. For example, the various representative tissue culture plate or tray 100, 200, 300, 400, 500, 600 embodiments may have substantially the same overall dimensions, including length, width, and height of a standard 96-well plate. A variety of equipment has been designed for use with such standard 96-well plates. When the various representative tissue culture plate or tray 100, 200, 300, 400, 500, 600 embodiments are fabricated with substantially the same dimensions as those of the standard 96-well plate format, the representative tissue culture plate or tray 100, 200, 300, 400, 500, 600 embodiments are capable of being used with the equipment designed for the 96-well format. In other embodiments, however, the various representative tissue culture plate or tray 100, 200, 300, 400, 500, 600 embodiments may have different dimensions, including those larger or smaller than a standard 96-well plate, such as 48-well, 24-well, 6-well, etc., or those that differ in any length, width, or height dimensions from a standard 96-well plate.

As discussed in greater detail below, the representative first, second, and third tissue culture plates or trays 100, 200, 300 differ from the fourth, fifth and sixth representative tissue culture plates or trays 400, 500, 600 primarily with respect to the type of channel 110, 210 utilized. The representative first, second, and third tissue culture plates or trays 100, 200, 300 utilize an “open” or U-shaped channel 110, which is open at the top or upper surface 105 along its entire length and width (openings 172), although in other embodiments, the channels 110 may only be partially open along their lengths and widths. The fourth, fifth and sixth representative tissue culture plates or trays 400, 500, 600 utilize a partially “closed” or tubular-shaped channel 210, which is open at the top or upper surface 105 only at the first and second ends 265, 270 of the channel 210 (openings 285), and which is otherwise not open to the top or upper surface 105 along the balance of its length and width, as illustrated, with the dashed lines in FIGS. 8-11 indicating the extension of the channels 210 below the upper surface 105 of the plate base 115. Additional variations will be apparent to those having skill in the art, such as providing additional holes from the top or upper surface 105 into the channels 210 (not separately illustrated), for example and without limitation, and any and all such variations are considered equivalent and within the scope of the disclosure.

A representative tissue culture plate or tray 100, 200, 300, 400, 500, 600 may further comprise a lip or skirt 120 arranged or disposed around the perimeter of the tissue culture plate or tray 100, 200, 300, 400, 500, 600, such as to increase the rigidity of the tissue culture plate or tray 100, 200, 300, 400, 500, 600 and/or to mate with an optional cover or lid 130, illustrated in cross-section in FIG. 7. Such a cover 130 (which may be lugged) is typically utilized to cover the tissue culture plate or tray 100, 200, 300, 400, 500, 600 and thereby avoid contamination of the contents of the channels 110, 210 of the tissue culture plate or tray 100, 200, 300, 400, 500, 600. Such a cover 130 may be fabricated as known or becomes known in the art, and may be fabricated to conform to the number, size, configuration and shape of the channels 110, such as to only cover the first and second ends 265, 270, such as in the various tubular embodiments, rather than cover the entire length of the open channels 110A, 110B, 110C, or to cover only some (but not all) of the various openings 285 (e.g., first and second ends 265, 270 of the various tubular embodiments, or partially cover the various open channels 110, all for example and without limitation). Additionally, when uncovered, the channels 110 may be open to the atmosphere along their length and width, or in the case of channels 210, at each end 265, 270.

The various representative tissue culture plate or tray 100, 200, 300, 400, 500, 600 embodiments may be comprised of a wide variety of materials, many of which are described in greater detail below, such as any of various plastics, polymers, natural and synthetic rubber materials, glass, ceramics, and other silicon or silica-derived materials and products, for example and without limitation. The tissue culture plates or trays 100, 200, 300, 400, 500, 600 and covers 130 can be manufactured from a variety of materials, such as HTM140, Helios, and simulated polypropylene, and assembled in a sterile environment, for example and without limitation. In addition, the various representative tissue culture plate or tray 100, 200, 300, 400, 500, 600 embodiments may be manufactured or fabricated as known or becomes known in the relevant arts of standard 96-well plate and Petri dish fabrication, including molding, stamping, thermoforming, injection molding, 3D printing, machining, and so on, also for example and without limitation. The various representative tissue culture plate or tray 100, 200, 300, 400, 500, 600 embodiments also may be manufactured or fabricated as a single part or as multiple parts which are joined together, for example and without limitation. For example, tissue culture plates or trays 400, 500, 600 may be fabricated as two parts and joined together (along the dashed line 405 illustrated in FIG. 8). The representative tissue culture plate or tray 100, 200, 300, 400, 500, 600 may be optically or visually opaque, or it may be substantially clear or otherwise optically transmissive, at any of various light and sound wavelengths (e.g., visual spectrum, X-ray spectrum, ultrasound spectrum, etc.), to allow better microscopic visualization of cells 150 and magnetic particles 70 in the channels 110, 210, including fluorescent microscopy, for example and without limitation. Any and all such variations are considered equivalent and within the scope of the disclosure.

The representative tissue culture plate or tray 100, 200, 300, 400, 500, 600 comprises a plate base 115 having a top surface 105 and a bottom surface 125, and further having a plurality of lateral sides (or edges), illustrated as comparatively longer sides 106 and 108 and comparatively shorter sides 112 and 114. In representative embodiments, the longer sides 106 and 108 are parallel to each other, and the comparatively shorter sides 112 and 114 are parallel to each other, with the longer sides 106 and 108 generally perpendicular (α=90°) to the comparatively shorter sides 112 and 114, to define an overall elongated cuboid form factor for the plate base 115. In addition, one of the comparatively shorter sides, illustrated as the side 112, is also chamfered or beveled to form additional angled sides 116, 118 (illustrated at equal offset angles of 135° (β=135°), although any offset angles may be utilized equivalently), to form a plate base 115 having a form of a truncated or chamfered elongated cuboid. The plate base 115 comprises a plurality of cavities (or voids), which for the selected embodiments, are formed as elongated cavities or tubes, effectively forming channels (troughs, grooves or tubes) 110, 210 (or, equivalently, depressions, valleys, bores, gaps, orifices, hollows, slits, tubes, or passages), with each channel 110, 210 having a depth 155, a length 135, and a width 145, with the width 145 being shorter than the length 135 of the channel 110, 210 (the length 135 is longer or greater than the width 145, in accordance with customary usage), and with the channels 110, 210 being spaced apart from the plurality of sides 106, 108, 112, 114 by one or more predetermined distances, which may be the same or different, and which may be symmetric or asymmetric (e.g., the spacing of the outermost channels 110, 210 from the longer sides 106 and 108 may be the same or different than the spacing of the channels 110, 210 from the comparatively shorter sides 112 and 114 and/or angled sides 116, 118.

The channels 110, 210 may have any selected or predetermined depth 155, and the depth 155 may vary along the length 135 and/or width 145 of the channel 110, 210. The width 145 (and/or length 135) of the channel 110, 210 may be variable (as illustrated in FIGS. 3, 4, 10 and 11) and may correspond to an approximate dimension of a structure being modeled; for example, the various representative tissue culture plate or tray 100, 200, 300, 400, 500, 600 embodiments may be used for modeling linear or curvilinear movement of particles within a human cerebral artery. In that case, the channels 110, 210 would have a width of from three to five mm, as those are the typical dimensions of a human cerebral artery (e.g., a larger width or diameter to model a larger vessel, such as a carotid artery, and a smaller width or diameter to model a smaller vessel, such as a more distal brain vessel). Such changes in width 145 may be successive (e.g., a smooth or continuous variance) or incremental (e.g., a stepped variance). In the representative tissue culture plate or tray 100, 400 illustrated in FIGS. 1 and 8, the length 135 of the channels 110, 210 may be and typically are about 10.5 cm long, for example and without limitation. The top surface 105 of the representative tissue culture plate or tray 100, 200, 300, 400, 500, 600 may extend between the sides 106, 108, 112, 114 and the periphery 124 of the channels 110, 210. The channels 110, 210 may be spaced apart from each other by any selected or predetermined distance, depending on the selected embodiment.

A representative tissue culture plate or tray 100, 400 illustrated in FIGS. 1 and 8 has been fabricated which may be utilized in a standard 96-well plate reader, with a length of 127.8 mm, a width of 85.5 mm, and a height of 13.04 mm. Eight channels 110 were formed, with each channel 110 separated by 9 mm center-to-center, and a width of 3.175 mm, with outermost channels 110 arranged 10.87 mm (distance 147, FIG. 2) from the shorter sides 112, 114 and 9.485 mm from the longer sides 106, 108 (distance 137, FIG. 2). In addition, as illustrated in the various Figures, chamfers (8 mm from each side) may be utilized (e.g., on two corners) to create a truncated, chamfered or beveled rectangular cuboid or slab (with the angled sides 116, 118 then each being 8·√2 mm in length). The width of the lip or skirt 120 was 2 mm around the periphery 122 of the plate base 115, to conform to and allow use of a common 96-well lid.

While the channels 110A, 210A are illustrated in FIGS. 1, 2, 8 and 9 as substantially parallel, spaced apart equally from each other, and oriented in the same direction for the representative tissue culture plate or tray 100, 400, as a representative type of channel 110, 210, those having skill in the art will recognize that innumerable variations are available, including length 135, depth 155 and width 145 of the channels 110, 210, channel orientation (e.g., straight, curvilinear, wavy, sinusoidal, branched, etc.), spacing variations, type or shape of the channel 110, 210 (e.g., semicircular, semi-elliptical, circular, elliptical), etc., and any and all such variations are considered equivalent and within the scope of the present invention. In addition, as illustrated using dotted lines in FIG. 2A, any two or more channels 110, 210 of the plurality of channels 110, 210 may coupled or connected to each other (e.g., in fluid or particle communication), connected to each other using additional cavities or channels 153 (having any selected width or depth), in addition to the illustrated couplings of the branch sections 195 to each other.

For example, and without limitation, the channels 110, 210 may run parallel to the length of the tissue culture plate or tray 100, 400 (as illustrated), or parallel to the width of the plate (not separately illustrated), or at an offset angle from the length or width, such as in the various branching configurations illustrated and discussed below with reference to FIGS. 3, 4, 10, and 11. The channels 110, 210 may run parallel to one another as illustrated for the tissue culture plate or tray 100, 400, or they may extend at angles from one another, as illustrated for the tissue culture plates or trays 200, 300, 500, 600. The channels 110, 210 or channel sections 195 may intersect with one another, or one channel 110, 210 may branch into a plurality of channels 110, 210 or channel sections 195, also as illustrated for the tissue culture plates or trays 200, 300, 500, 600. The channels 110, 210 or channel sections 195 may extend in a straight line (e.g., in the tissue culture plate or tray 100, 400) or they may angle or curve (e.g., in the tissue culture plate or tray 200, 300, 500, 600). The channels 110, 210 or channel sections 195 may all be of the same dimensions, including length, width, and depth, or the dimensions may vary from one channel 110, 210 or channel section 195 to another. The channels 110, 210 may extend substantially from one end of the tissue culture plate or tray 100, 200, 300, 400, 500, 600 to the opposite end, or they may extend only a short portion of the distance from one end of the tissue culture plate or tray 100, 200, 300, 400, 500, 600 to the opposite end.

The location of the channels 110, 210 or channel sections 195 may correspond to the location of rows or columns of wells in a standard 96-well plate or other standard plate such as 48-well, 24-well, 6-well, etc., for example and without limitation. In particular, a plurality of the channels 110, 210 may be centered along the space where a row or column of wells would be located in a standard 96-well plate. Thus, the tissue culture plate or tray 100, 200, 300, 400, 500, 600 could be used with a standard 96-well plate reader, for example and without limitation, such as for microscopy, color detection, fluorescence detection, etc., also for example and without limitation. In other embodiments, the location of the channels 110, 210 or channel sections 195 may be completely customize to correspond to the conduits (naturally occurring or implanted) of an individual patient (e.g., by importing patient data into a CAD system and fabricating a tissue culture plate or tray 100, 200, 300, 400, 500, 600 using known 3-D printing, for example and without limitation), such as for advance modeling of magnetic particle treatment alternatives.

Various representative tissue culture plate or tray 200, 300, 500, 600 embodiments are illustrated in FIGS. 3, 4, 10, and 11 having additional forms, such as branched configurations of the channels 110B, 110C, 210B, 210C, with a plurality of branch sections 195 branching or dividing from a central (or main) channel (or channel branch section) 198. As illustrated for the representative tissue culture plate or tray 200, 500 embodiments, the channels 110B, 210B have generally linear (straight) branch sections 195A, and successively branch at any selected angle at any of a plurality of branch points 185, starting from the central channel (or channel branch section) 198. As illustrated for the representative tissue culture plate or tray 300, 600 embodiments, the channels 110C, 210C have generally curved or curvilinear (straight) sections 195B, and also successively branch at any selected angle at any of the plurality of branch points 185, also starting from the central channel (or channel branch section) 198. Also as illustrated in FIGS. 3, 4, 10, and 11, the channels 110B, 110C, 210B, 210C have a variable width, successively branching into channel sections 195A, 195B having progressively smaller widths.

The various channels 110, 210 may have any of a plurality of different configurations and surface textures, as illustrated in FIGS. 5, 6, 13 and 14. Referring to FIG. 5, a channel 110 has side walls 170A which are substantially perpendicular (as an example) to the top surface 105 and which extend a depth 175, which then curve or arc (having a semi-circular or U-shaped arc with a radius 165A, for this tissue culture plate or tray 100 embodiment) to form a semi-circular channel bottom 180A having a comparatively smooth surface 190A. In representative embodiments, the side walls 170A are optional and may be omitted, with use of the side walls 170A depending upon the selected depth 155 and width 145 of the channels 110 and the selected configuration of the channel bottom 180. Such a semi-circular channel bottom 180A is particularly suitable for simulating vasculature, other vessels, catheters, etc., for example and without limitation.

Referring to FIG. 13, a channel 210 has side walls 170B which are substantially circular (as an example), arranged below the top surface 105 and extending to a depth 155, which are curved (having a circular-shaped arc with a radius 165B, for this tissue culture plate or tray 400 embodiment) to form a circular (in cross-section) channel 210A having a comparatively smooth surface 190A. Such a circular channel 210A is particularly suitable for simulating vasculature, other vessels, catheters, etc., for example and without limitation.

For example, as shown in the various Figures, in a representative embodiment, the cross-section of the channels 110 may be semi-circular or U-shaped, as shown in FIG. 5. In other embodiments, the channels 110 may have side walls 170A that meet at angles, such as sides that form a square or rectangular cross-section (channel 110G). Also in other embodiment, the channels 110 may have a polygonal cross-section, comprised of more or fewer than three sides, and wherein the sides meet at non-90 degree angles (e.g., channels 110E, 110H, 110J, 110 L).

Also for example, as shown in the various Figures, in a representative embodiment, the cross-section of the channels 210 may be circular (channel 210A), as shown in FIG. 13. In other embodiments, the channels 210 may have side walls 170B that meet at angles, such as sides that form a square or rectangular cross-section (channel 210F). Also in other embodiment, the channels 210 may have a polygonal cross-section, comprised of more or fewer than three sides, and wherein the sides meet at non-90 degree angles (e.g., channels 210E, 210G, 210J, 210 M).

Referring to FIG. 6, a variety of different configurations and surface textures are shown, for example and without limitation: a channel 110D having a semicircular channel bottom 180A having a textured (comparatively non-smooth) surface 190B; a channel 110E having a triangular channel bottom 180B having a comparatively smooth surface 190A; a channel 110F having a semi-elliptical channel bottom 180C having a comparatively smooth surface 190A; a channel 110G having a flat channel bottom 180D (i.e., square or rectangular in cross-section) having a comparatively smooth surface 190A; a channel 110H having a polygonal channel bottom 180E having a comparatively smooth surface 190A; a channel 110J having a triangular channel bottom 180B having a textured (comparatively non-smooth) surface 190B; a channel 110K having a semi-elliptical channel bottom 180C having a textured (comparatively non-smooth) surface 190B; and a channel 110L having a polygonal channel bottom 180E having a textured (comparatively non-smooth) surface 190B. Any of the various representative tissue culture plate or tray 100, 200, 300 embodiments may utilize any and all such variations of channel 110 configurations and surface textures, in any number, permutation and/or combination within a tissue culture plate or tray 100, 200, 300, and all of which are considered equivalent and within the scope of the present invention.

Referring to FIG. 14, a variety of different configurations and surface textures are shown, for example and without limitation: a channel 210D being elliptical in cross-section and having a comparatively smooth surface 190A; a channel 210E being triangular in cross-section and having a comparatively smooth surface 190A; a channel 210F being square or rectangular in cross-section and having a comparatively smooth surface 190A; a channel 210G being polygonal in cross-section and having a comparatively smooth surface 190A; a channel 210H being circular in cross-section and having a textured (comparatively non-smooth) surface 190B; a channel 210J being elliptical in cross-section and having a textured (comparatively non-smooth) surface 190B; a channel 210K being triangular in cross-section and having a textured (comparatively non-smooth) surface 190B; and a channel 210L being square or rectangular in cross-section and having a textured (comparatively non-smooth) surface 190B (another channel 210 (not separately illustrated) may also be polygonal in cross-section and having a textured (comparatively non-smooth) surface 190B). Any of the various representative tissue culture plate or tray 400, 500, 600 embodiments may utilize any and all such variations of channel 210 configurations and surface textures, in any number, permutation and/or combination within a tissue culture plate or tray 400, 500, 600, and all of which are considered equivalent and within the scope of the present invention.

The surface of the channels 110, 210 may be coated or etched, or both, including coating with any of various materials. For example and without limitation, a coating 140 may be applied to the channels 110, 210 or to the channel bottoms 180, such as the various cell adhesion coatings utilized to adhere cells for tissue culture. For example and without limitation, the culture adhesive applied as a type of coating 140 may be any substance or substances that facilitate(s) cell attachment to a material of the channel 110, 210, such as poly-lysine, poly-ornithine, collagen, laminin, matrigel, and a combination thereof. The culture adhesive applied as a type of coating 140 may be applied at any suitable concentration, and may be diluted to a desired concentration prior to use. Also for example and without limitation, etching may be applied to the channels 110, 210 or to the channel bottoms 180, to create a textured (comparatively non-smooth) surface 190B. The coating 140 or textured (comparatively non-smooth) surface 190B may facilitate the ability of the cells 150 to adhere to the surface of the channels 110, 210.

The channels 110, 210 may be used with or without cells 150. When used with cells 150, the cells may be human or animal cells. Representative cell types used in the channels 110, 210 may include for example and without limitation: vascular endothelial cells, cancer cells, stem cells, cell spheroids, or combinations of cells.

The channels 110, 210 and/or channel bottoms 180 may be filled, completely or partially, with a medium 160 such as serum, lymph, artificial CSF, clotted or unclotted blood, or urine, also for example and without limitation. For example, a blood clot and/or cells may be deposited in any selected location along a channel 110, 210 or channel segment 195, while a liquid medium such as serum may extend throughout a channel 110, 210. A tissue or cell culture may be performed in the channels 110, 210. Cells 150 may be cultured along the entire length, channel bottoms 180 and/or side walls 170 of the channels 110, 210. Cells 150 may be grown in the tissue culture plates or trays 100, 200, 300, 400, 500, 600 using standard tissue culture technique (e.g., 37° C., humidified atmosphere, 5% CO₂), including initial cell seeding in the laminar flow hood.

After the tissue culture is complete, the medium 160 in the channels 110, 210 may be changed to another medium 160 or fluid. Magnetic particles 70 may be added to at least one of the channels 110, 210. The magnetic particles 70 may be in aliquots. The magnetic particles 70 may be added at one end (e.g., 265) of the at least one channel 110, 210 or they may be added at a location a distance from an end (e.g., 265, 270) of the channel 110, 210. The magnetic particles 70 may be moved magnetically, as discussed in greater detail below. Movement of magnetic particles 70 in the tissue culture plates or trays 100, 200, 300, 400, 500, 600 can be observed visually or using any of the imaging techniques mentioned above, for example and without limitation. Velocity and uptake can be measured, and cells 150 can be harvested for further analysis. Cells 150 in tissue culture plates or trays 100, 200, 300, 400, 500, 600 can be observed using a standard inverted light microscope, also for example and without limitation. Thus tissue culture plates or trays 100, 200, 300, 400, 500, 600 comprising a substantially optically transmissive or transparent material may be desirable for some applications.

Blood clots may be established within portions of the channels 110, 210, for the study of therapeutic clot lysis, for example.

As an option, and as illustrated in FIGS. 1 and 8, a representative tissue culture plate or tray 100, 200, 300, 400, 500, 600 may also include indicia 260, such as a marked rule for distance measurement. The indicia 260 may be added (e.g., ground or scribed) into or arranged or integrally-formed with the top surface 105 or elsewhere on the representative tissue culture plate or tray 100, 200, 300, 400, 500, 600, for example. Also for example, the indicia 260 may also include other markings useful for measurements, such as numberings, and may provide any selected resolution or line spacing.

FIG. 16 is an isometric view of a representative first tissue culture plate or tray 100 embodiment having tubes 340, cylinders 355 and monitors 345 arranged in various channels 110A. As illustrated in FIG. 16, use of such tubes 340 (or cylinders 355) within a channel 110 allow for additional types of study and monitoring, such as for modeling movement of magnetic particles through catheters or stents (both of which may be placed inside of other vessels), or using solid cylinders 355 to model movement around the spinal cord in the subarachnoid space. For example, such tubes 340 may be a shunt catheter, such as for ventriculoperitoneal shunting of CSF, allowing testing of the movement of magnetic particles under different conditions, such as if the catheter is obstructed with a blood clot, for example and without limitation. Monitors 345, which may be magnetic monitors or fluorescent monitors, or monitors for convective flow and pulsation, for example and without limitation, may also be useful for providing monitoring of the movement of magnetic particles in conditions of darkness, such as found within the human body.

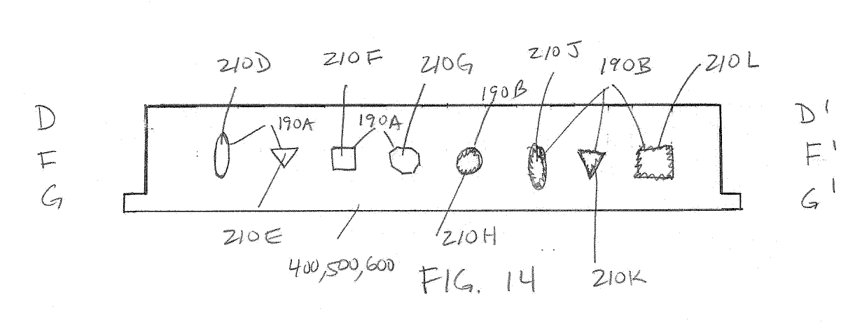

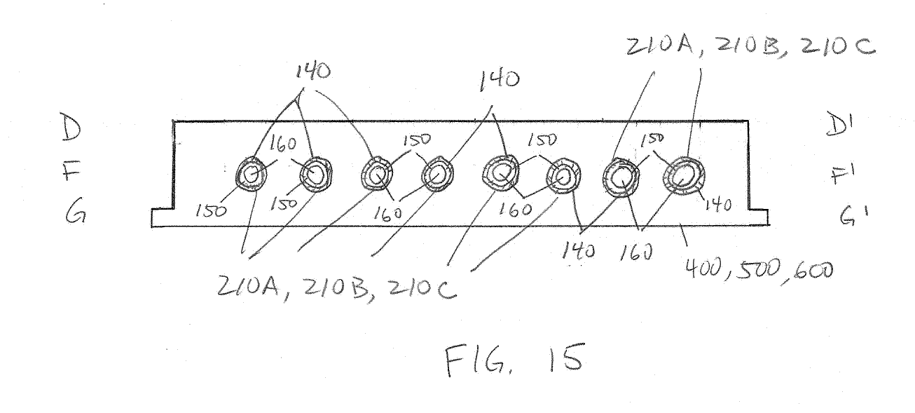

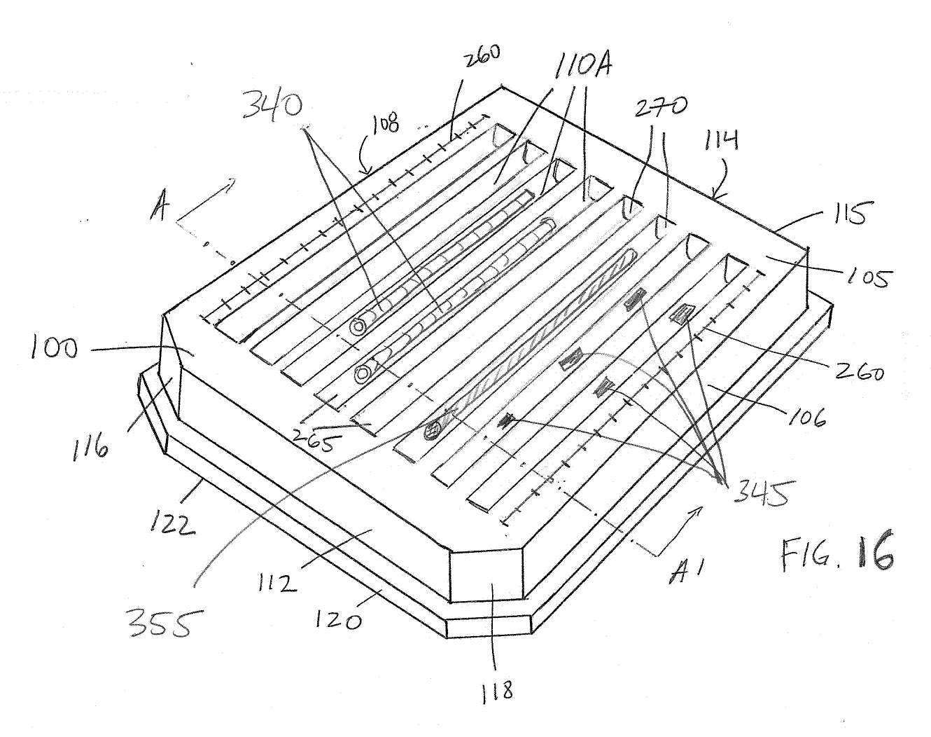

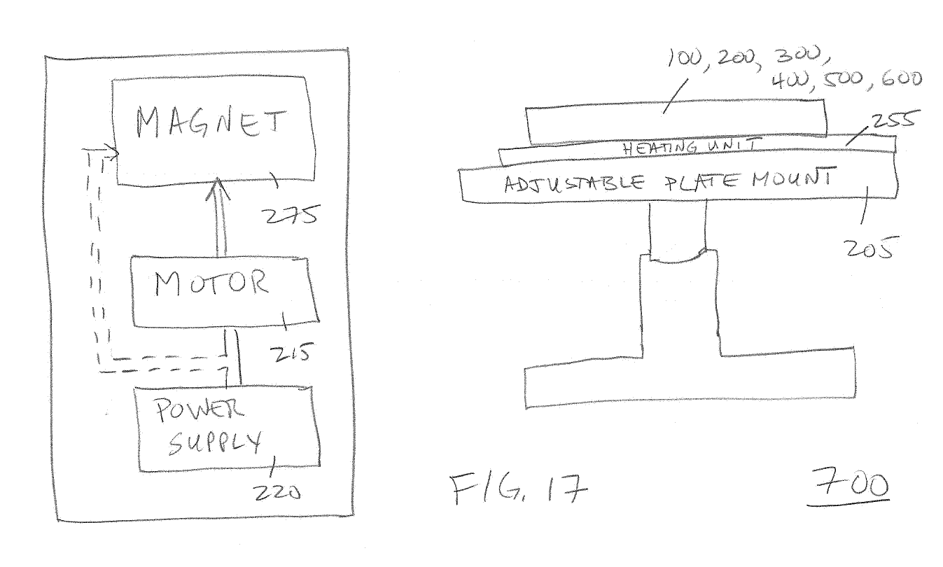

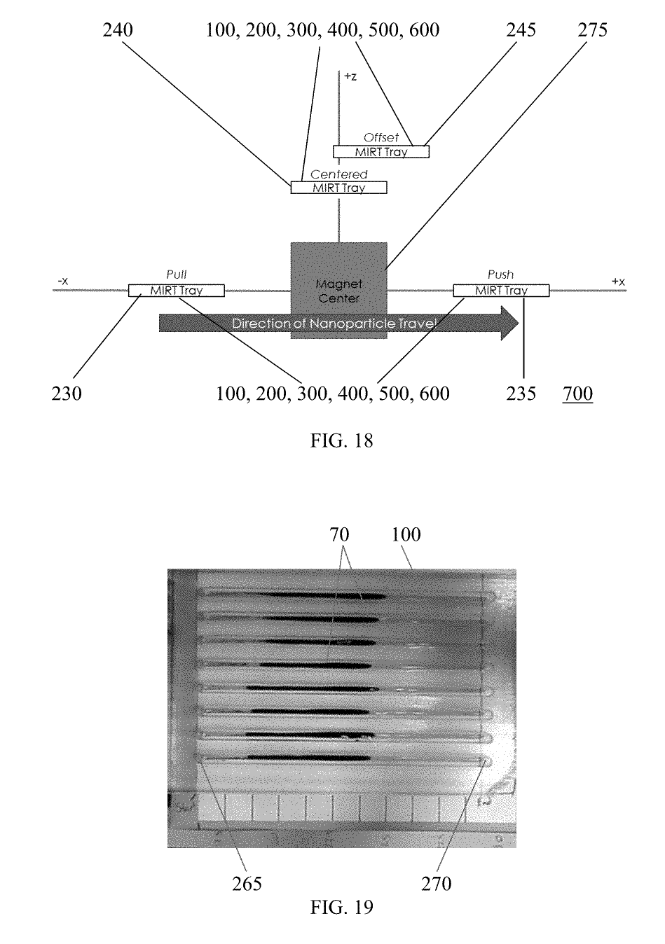

FIG. 17 is a block diagram and elevational view of a representative tissue culture plate or tray system 700 embodiment. FIG. 18 is a block diagram and elevational view of a representative tissue culture plate or tray system 700 embodiment, illustrating representative placement positions of a representative tissue culture plate or tray 100, 200, 300, 400, 500, 600 embodiment with respect to a magnet. FIG. 19 is a photograph of the representative first tissue culture plate or tray 100 embodiment of FIG. 1 showing linear translational displacement of MNPs following application of a rotating magnetic field. Varying and/or smaller volumes of magnetic particles may also be utilized.

The representative system 700 implements magnetically-induced rotation and translation and comprises a magnetic device 250 and a tissue culture plate or tray 100, 200, 300, 400, 500, 600. The channels 110, 210 will contain the magnetic particles (or beads) 70. The tissue culture plates or trays 100, 200, 300, 400, 500, 600 are designed to facilitate the use of a wide variety of magnetic particles (or beads) 70, and any type of magnetic particles 70 may be utilized. The magnetic particles (or beads) 70 may consist of magnetite cores (˜75 nm), and may have a thin, pharmaceutical grade PEG-coating, for example and without limitation, such as those which may be fabricated in any laboratory or other facility (e.g., for customization) or obtained commercially from FullScaleNANO, Inc. (https://www.fullscalenano.com), of Tallahassee, Fla. USA; Thermofisher Scientific of Pittsburgh, Pa. USA, or from any other supplier, also for example and without limitation. The magnetic device 250 comprises a magnet 275, an optional motor 215 (to rotate the magnet 275, depending upon the type of magnet utilized), and a power supply 220. Other typical components, such as switches, magnetic shielding, are not separately illustrated. A representative magnetic device 250 (referred to as a “mini-MED”) may be obtained commercially from Pulse Therapeutics, Inc., of St. Louis, Mo. (https://www.pulsetherapeutics.com), and is also described in: U.S. Pat. No. 8,529,428; U.S. Pat. No. 8,313,422; and U.S. Pat. No. 8,308,628; each of which is incorporated herein by reference in its entirety with the same full force and effect as if set forth in its entirety herein. An adjustable plate mount 205 may be provided for positioning the tissue culture plates or trays 100, 200, 300, 400, 500, 600 at various distances (laterally and elevationally) from the magnet 275, such as at the various distances illustrated in FIG. 18. A heating unit 255 may be provided for use in maintaining the temperature the fluid and/or cells. Other components, such as control components (e.g., switches and rotational speed control), are not separately illustrated.

The magnet 275, when embodied as a permanent magnet, is rapidly rotated by the motor 215. The magnet 275 may be embodied as other types of magnets as well, including solenoid or other electromagnets, which do not require physical rotation to generate a rotating magnetic field (e.g., to change the amplitude and polarity of the magnetic field), such that no motor 215 is required. The rotating magnetic field may have any magnitude suitable for the selected magnetic particles 70, and causes magnetic particles (or beads) 70 (such as aggregates of MNPs) to counter-rotate (like meshed gears) at physiologic distances (i.e., 20 cm) even through bone, muscle, blood vessels, and other intervening structures. For example, the magnetic field for rotating and moving magnetic particles 70 may be generated by a neodymium-boron-iron permanent magnet (2 inch neodymium cube, grade N52, BrMax: 14800 gauss, from Applied Magnets, Plano, Tex., USA), and the magnet 275 may be rapidly rotated (e.g., 3 Hz), causing magnetic particles 70 to counter-rotate and move by means of surface traction. Any suitable type of magnet 275, motor 215, power supply 220 and other components which are known or which become known in the electrical and electronic arts may be utilized in the system 700.

The system 700 using MIRT is able to move the magnetic particles (or beads) 70 at a distance along tubes (e.g., vessels) and across surfaces, even when tissue (such as bone, muscle, and/organs such as the brain) intervenes between the magnetic particles (or beads) 70 and source magnet 275. Potential medical applications for MIRT are numerous, as mentioned above. Whether or not these applications will be hindered by factors affecting wall shear and particle adherence to the surface (or lumen), among many other factors, may need to be determined.

A tissue culture plate or tray 100, 200, 300, 400, 500, 600 may have innumerable positions relative to the magnet 275, with FIG. 18 illustrating four representative positions 230, 235, 240, and 245, as examples. Referring to FIG. 18, the typical direction of translational motion of the magnetic particles (or beads) 70 is in the +x direction (left to right in FIG. 18), assuming a counterclockwise rotation of the magnet 275 and a clockwise rotation of the magnetic particles (or beads) 70. As illustrated, one or more representative tissue culture plates or trays 100, 200, 300, 400, 500, 600 may be arranged or disposed, relative to the magnet 275: (1) in a first position 230 (a “pull” position), in which magnetic particles (or beads) 70 deposited at a first end 265 of a channel 110, 210 of a representative tissue culture plate or tray 100, 200, 300, 400, 500, 600 will be pulled toward the second end 270 of the channel 110, 210 of the representative tissue culture plate or tray 100, 200, 300, 400, 500, 600; (2) in a second position 235 (a “push” position), in which magnetic particles (or beads) 70 deposited at a first end 265 of a channel 110, 210 of a representative tissue culture plate or tray 100, 200, 300, 400, 500, 600 will be pushed toward the second end 270 of the channel 110, 210 of the representative tissue culture plate or tray 100, 200, 300, 400, 500, 600; (3) in a third position 240 (a centered position), directly above the magnet 275 (or directly below the magnet 275, not separately illustrated), in which magnetic particles (or beads) 70 deposited at a first end 265 of a channel 110, 210 of a representative tissue culture plate or tray 100, 200, 300, 400, 500, 600 will be initially pulled and then pushed toward the second end 270 of the channel 110, 210 of the representative tissue culture plate or tray 100, 200, 300, 400, 500, 600; (4) in a fourth positions 245 (as an “offset” position), in which magnetic particles (or beads) 70 deposited at a first end 265 of a channel 110, 210 of a representative tissue culture plate or tray 100, 200, 300, 400, 500, 600 will be pushed toward the second end 270 of the channel 110, 210 of the representative tissue culture plate or tray 100, 200, 300, 400, 500, 600.

It should be noted that the direction of rotation of the magnet 275 (or magnetic field) can be reversed to reverse or remove the magnetic particles 70, or stopped once the magnetic particles 70 reach the desired location. The magnetic particles 70 may also be moved back and forth through a region multiple times, also through forward and reverse directions of rotation of the magnet 275 and/or magnetic field.

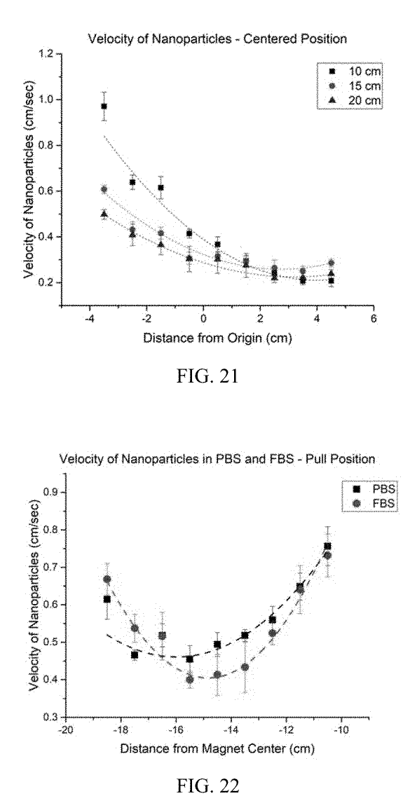

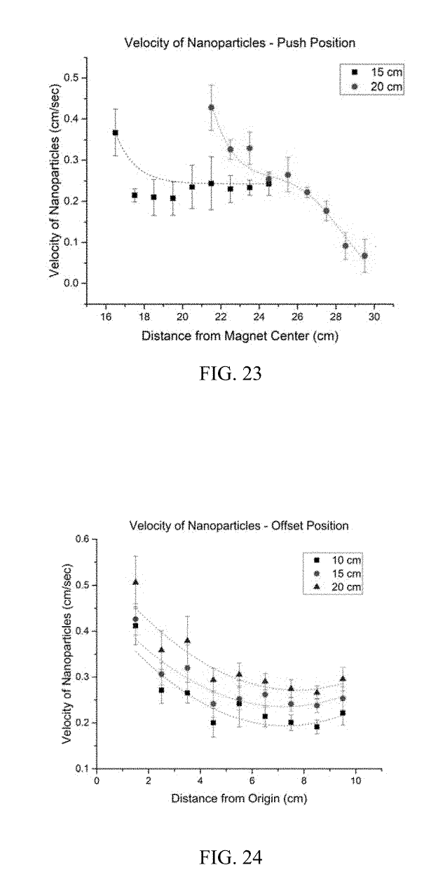

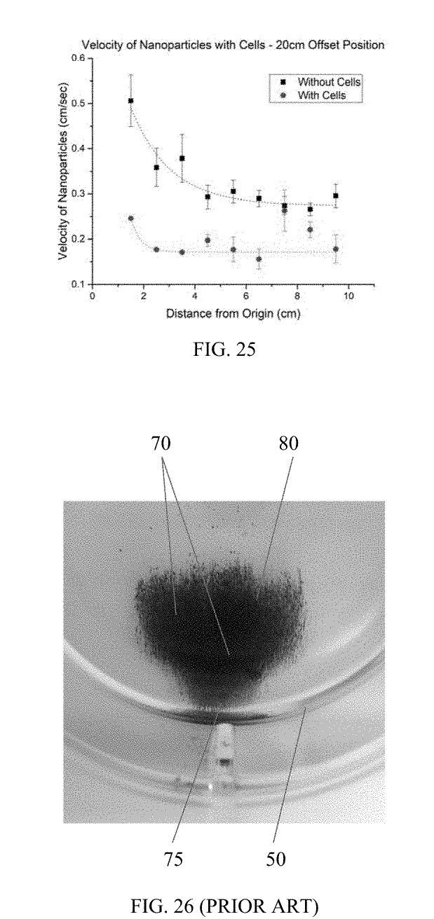

Representative graphs of magnetic particle 70 velocity versus distance of a representative tissue culture plate or tray 100 to a magnet 275, and in the various relative positions described above, with and without cells 150, in a representative tissue culture plate or tray system 700 embodiment, are shown in FIGS. 19-25. For example, initial studies of these various positions have found that about a 20 cm distance between the magnetic particles (or beads) 70 and the magnet 275 may be optimum for highest velocity, and multiple factors will affect such determinations, as mentioned below. It should be noted, in FIGS. 19-25, that any reference to “distance from the origin” means distance from the center of the magnet 275.

Representative linear movement of magnetic particles (or beads) 70 is illustrated in FIG. 19, in which magnetic particles (or beads) 70 deposited at first ends 265 of several channels 110 of a representative tissue culture plate or tray 100 have been moved toward the second ends 270 of the channels 110 of the representative tissue culture plate or tray 100.

In an example, velocities of magnetic particles 70 were determined after filling channels 110A of a representative tissue culture plate or tray 100 having semicircular channel bottoms 180A with media 160 including phosphate-buffered saline (PBS), tissue culture medium with 10% serum (FBS), or 100% FBS at room temperature. Velocity determinations were made by timing the transit of the leading edge of the magnetic particles 70 along 10 cm of each channel 110A. In initial studies, the representative tissue culture plate or tray 100 was placed directly over the rotating magnet 275, at distances ranging from 7.5 to 30 cm. Five mm was used as the channel 110A width 145, and lanes were each filled with a volume of 2 ml. An aliquot of 100 uL of magnetic particles 70 per channel 110A was used (from the thoroughly vortexed 1× stock solution of 25 mg/ml). The magnetic particles 70 were aligned to the first side 265 of the representative tissue culture plate or tray 100 by running the magnet for 3 seconds.

Human glioblastoma cells were maintained using standard tissue culture technique in Dulbecco's Modified Minimal Essential Medium (DMEM) with 10% fetal bovine serum (and antibiotics), before and after being seeded into the channels 110A of the representative tissue culture plate or tray 100. Cells were grown in the representative tissue culture plate or tray 100 in a humidified atmosphere with 5% CO2 at 37° C., and were studied using standard (inverted) light and fluorescence microscopy (EVOS FL Auto 2, Thermo Fisher Scientific, Waltham, Mass., USA). Proliferation of U87 cells in the channels 110A of the representative tissue culture plate or tray 100 was studied for a variety of initial seeding counts (50,000-200,000 cells), for up to 96 hours, with daily change of media. For further studies, an initial seeding count of 100,000 cells, with growth for 48 hours, was chosen. For velocity determinations with cells, a ⅜ inch channel 110A width 145, with a 20 uL dose of magnetic particles 70 (with cell-free control channels 110A) was used (with a distance above magnet: 10 cm). After growth in the representative tissue culture plate or tray 100, live cells were rinsed with PBS, and cell nuclei were stained with Hoechst 33342 (DNA stain for live cells), for 10 minutes.

Magnetic particle 70 aggregates could be readily moved along the fluid-filled channels 110A of the representative tissue culture plate or tray 100 by the mini-MED (which was stationary), at a variety of distances and positions. The composition of the representative tissue culture plate or tray 100, and factors such as the roughness of the channels 110A, were found to affect the adherence of the aggregates to the walls of the lanes.

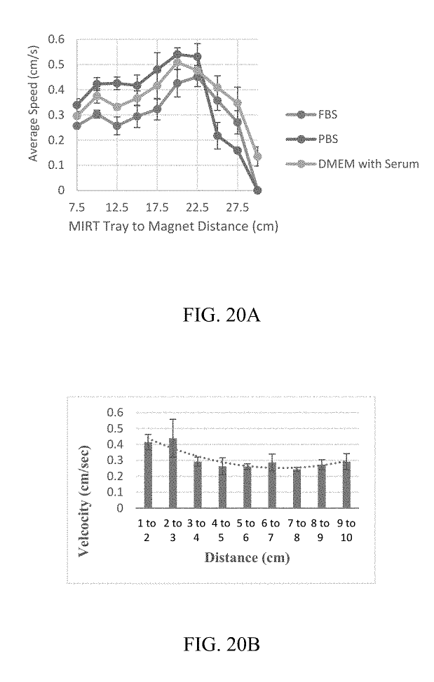

Velocity of the leading edge of the magnetic particles 70 was found to vary according to representative tissue culture plate or tray 100 position and solution type. In PBS, the maximum average magnetic particle 70 speed was 0.54+0.03 cm/sec at a distance of 20 cm above the magnet 275. A graph of the velocity results for PBS, DMEM with 10% serum, and 100% FBS, according to distance above the magnet 275, can be seen in FIG. 20A.

U87 cells grew readily in the channels 110A of the representative tissue culture plate or tray 100 with a variety of initial seeding counts. Addition of cells to the channels 110A of the representative tissue culture plate or tray 100 reduced the average magnetic particle 70 velocity by 40+5% at the position of 10 cm above the magnet. Such velocity determinations are dependent on positioning of the representative tissue culture plate or tray 100 with respect to the magnet 275.

Distance to the magnet 275, viscosity of the media 160, and particle adhesion to the (cellular) surface, would seem to be major factors affecting magnetic particle 70 (such as IONs) rotation and translation in response to the rotating magnetic field. Data shown here indicate that magnetic particles 70 are easily rotated and rapidly moved at physiologic distances in the system 700. As demonstrated with the representative tissue culture plate or tray 100, this phenomenon occurs even through 100% serum, and over a cellular monolayer. The characteristics of the representative tissue culture plate or tray 100 and other variables affecting movement of the magnetic particles 70 are described below.

The three-component system 700 of the magnetic device 275 generating a rotating magnetic field, magnetic particle 70, and the representative tissue culture plate or tray 100, is inexpensive, especially when compared to the cost of constructing magnetic systems capable of moving or attracting IONs at comparable distances (7.5-35 cm) without magnet rotation. Multiple copies of the representative tissue culture plate or tray 100 can be made to precise specifications. Alterations in these specifications may change magnetic particle 70 translation, cell imaging, and cellular adherence to the channels 110A of the representative tissue culture plate or tray 100. The parameters of the representative tissue culture plate or tray 100 can be varied according to the physiologic structure that is being modeled. Some representative conduits and surfaces within the body are described below. The channels 110A of the representative tissue culture plate or tray 100 may be filled with solutions that may differ in composition and viscosity (e.g., blood, cerebrospinal fluid, lymph). Multiple channels 110A facilitate making simultaneous determinations under varying conditions (such as drug and/or particle dose), with suitable controls. Flow and pulsations may be added to the system 700 to improve the accuracy of the modeling. 3D printing can also be performed with data imported from a specific patient, or averages from multiple patients (e.g., anatomy of the middle cerebral artery bifurcation).

Various possible factors may affect the movement and velocity of magnetic particles (or beads) 70 in the representative tissue culture plate or tray 100, 200, 300, 400, 500, 600, including, for example and without limitation: (1) nanoparticle and aggregate variables such as: particle type, size and composition; particle coating(s); particle mixtures (i.e., there may be a combination of types and various ratios); particle magnetization and zeta potential; particle aggregate size; particle and aggregate stability (e.g., potential disassembly due to torque, traction effects); the ratio of particle or aggregate size to channel 110, 210 width; adherence or friction between and among the magnetic particles (or beads) 70, the cells 150, and the walls 170 or channel bottom 180; torque; and prior magnetic exposure; (2) variables of the representative tissue culture plate or tray 100, 200, 300, 400, 500, 600 and channels 110, 210 such as: composition of the representative tissue culture plate or tray 100, 200, 300, 400, 500, 600; channel 110, 210 width, radius or diameter; surface texture 190, such as smoothness, pitting, dimpling, or irregularity (e.g., anisotropy); electrostatic effects; elasticity and/or flexibility of the representative tissue culture plate or tray 100, 200, 300, 400, 500, 600 and channels 110, 210; erosion and/or friability of the representative tissue culture plate or tray 100, 200, 300, 400, 500, 600 and channels 110, 210; coefficients of friction (static, dynamic, transitional); channel 110, 210 branching, branching patterns, bends or curves in any plane; channel 110, 210 coating(s) 140; and barrier phenomena (e.g., changes with encountering a wall, blood clot, aneurysm); (3) media 160 variables such as: pH; viscosity; fluid composition; fluid flow (volume, velocity, axial pulsation, meniscus and/or wall effects), with or against magnetic particle 70 movement; and webbing or matrix (e.g., arachnoid); (4) cellular variables (when cells may be grown in a representative tissue culture plate or tray 100, 200, 300, 400, 500, 600), such as: type of cell; cell density; adhesion of cells 150 to channel 110, 210 surfaces; adherence of magnetic particles (or beads) 70 to cell 150 surfaces; cellular uptake of magnetic particles (or beads) 70 (e.g., endocytosis, phagocytosis); cellular mixture (e.g., endothelial cells plus cancer cells); and ciliation; (5) magnetic source variables, such as: distance from the magnet 275; position with respect to the magnet 275; speed of rotation of the magnet 275; torque; and the strength and composition of the magnetic field, flux and/or gradient; and (6) miscellaneous factors such as temperature and external pulsation (e.g., perpendicular or normal to the channels 110, 210).

A wide variety of human and animal systems and structures may be modeled using a representative tissue culture plate or tray 100, 200, 300, 400, 500, 600 for MIRT including, for example and without limitation: (1) in the brain, spine and CNS: the ventricular system, the cerebral artery system, the cerebral venous system, the Virchow-Robbins spaces, and the spinal subarachnoid space; (2) in the genital-urinary tract: the bladder, kidney, ureter, fallopian tube, seminal tubule; and (3) generally throughout a human or animal body: the peritoneal cavity, the coronary arteries, veins and arteries generally, lymphatics, ducts (or biliary tree such as biliary ducts and pancreatic ducts), bronchioles, joints (knee, shoulder and hip), the retina, and the cornea.

As mentioned above, the representative tissue culture plate or tray 100, 200, 300, 400, 500, 600 is comprised of any one or more suitable materials, including combinations of materials of different types, and may include various additional coatings (in addition to the coatings 140 discussed above), such as a biocompatible or inert polymer or plastic, such as polycarbonate, latex, PMMA (poly(methyl methacrylate), PDMS (poly(dimethylsiloxane)), polystyrene or polytetrafluoroethylene (PTFE or Teflon), any type of biocompatible or inert glass, or any type of nonmagnetic biocompatible or inert metal or alloy, for example and without limitation. Representative types of biocompatible or inert glasses include, in addition to borosilicate glass (any silicate glass having at least 5% of boric oxide): soda-lime glass, a lead glass (including a lead-alkali glass), aluminosilicate glass (having aluminum oxide in its composition), ninety-six percent silica glass, and fused silica glass. Other representative examples of biocompatible or inert polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The biocompatible or inert polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof. For example, an Aclar® PVC film may be utilized.

The biocompatible or inert polymers may further include, but are not limited to, polyoleins (such as Thermanox®), or poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof. At least one of polymers can be a poly(ester amide), a poly(lactide) or a poly(lactide-co-glycolide) copolymer; and any derivatives, analogs, homologues, congeners, salts, copolymers and combinations thereof.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical, structural or magnetic coupling, connection or attachment, or adaptation or capability for such a direct or indirect electrical, structural or magnetic coupling, connection or attachment, including integrally formed components and components which are coupled via or through another component.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

It is claimed:

1. A tissue culture plate or tray for magnetically induced rotational and translational movement of magnetic particles, the tissue culture plate or tray comprising:
a plate base having an upper surface and a plurality of sides, the plate base having a form factor of a truncated cuboid; and
a plurality of channels arranged in the plate base, each channel of the plurality of channels spaced apart from one or more sides of the plurality of sides, each channel of the plurality of channels having a predetermined depth from the upper surface of the plate base, and each channel of the plurality of channels further having a length and a width, wherein the length is greater than the width.

2. The tissue culture plate or tray of claim 1, wherein the plurality of channels are arranged either parallel to each other or as a plurality of branches of a central channel of the plurality of channels.

3. The tissue culture plate or tray of claim 2, wherein the plurality of channels conform to a human or animal morphology.

4. The tissue culture plate or tray of claim 1, wherein the plurality of channels are arranged as a plurality of straight or curved branches or both straight and curved branches of a central channel of the plurality of channels, the central channel having a first width or diameter and the plurality of branching channels having second or more widths or diameters which are successively or incrementally smaller than the first width or diameter.

5. The tissue culture plate or tray of claim 1, wherein at least one channel of the plurality of channels has a smooth surface texture.

6. The tissue culture plate or tray of claim 1, wherein at least one channel of the plurality of channels has a non-smooth surface texture.

7. The tissue culture plate or tray of claim 1, wherein at least one channel of the plurality of channels has a varying width or depth.

8. The tissue culture plate or tray of claim 1, wherein at least one channel of the plurality of channels has a width from one (1.0) mm to ten (10) mm.

9. The tissue culture plate or tray of claim 1, wherein at least two channels of the plurality of channels are coupled to each other.

10. The tissue culture plate or tray of claim 1, wherein the plate base is comprised of a material compatible with an imaging technique selected from the group consisting of magnetic resonance imaging, x-ray imaging, computed tomography (CT) imaging, ultrasound imaging, light microscopy, fluorescence microscopy, and combinations thereof.

11. The tissue culture plate or tray of claim 1, wherein the plate base has an overall form factor matching a standard 96-well culture plate, or a standard 96-well culture plate, or a standard 48-well culture plate, or a standard 16-well culture plate, or a standard 6-well culture plate.

12. The tissue culture plate or tray of claim 1, wherein the plate base further comprises:
a skirt arranged around a periphery of the plate base.

13. The tissue culture plate or tray of claim 1, wherein the truncated cuboid defines a hexagon in the lateral dimension.

14. The tissue culture plate or tray of claim 1, wherein at least one channel of the plurality of channels is at least partially open to the upper surface of the plate base.

15. The tissue culture plate or tray of claim 1, wherein at least one channel of the plurality of channels is open to the upper surface of the plate base only at a first channel end and at one or more second channel ends, and wherein the region of the at least one channel between the first and second channel ends is closed to the upper surface of the plate base.

16. The tissue culture plate or tray of claim 1, wherein at least one channel of the plurality of channels has a semicircular channel bottom.

17. The tissue culture plate or tray of claim 1, wherein at least one channel of the plurality of channels is open to the upper surface of the plate base and has a bottom surface having a cross-sectional shape selected from the group consisting of: semi-circular, square, rectangular, semi-elliptical, polygonal, triangular, and combinations thereof.

18. The tissue culture plate or tray of claim 1, wherein at least one channel of the plurality of channels is tubular.

19. The tissue culture plate or tray of claim 1, wherein at least one channel of the plurality of channels is tubular and has a cross-sectional shape selected from the group consisting of: circular, square, rectangular, elliptical, polygonal, triangular, and combinations thereof.

20. A tissue culture plate or tray for magnetically induced rotational and translational movement of magnetic particles, the tissue culture plate or tray comprising:
a plate base having an upper surface and a plurality of sides, the plate base having a form factor of a truncated cuboid, wherein the plate base is comprised of a material compatible with an imaging technique selected from the group consisting of magnetic resonance imaging, x-ray imaging, computed tomography (CT) imaging, ultrasound imaging, light microscopy, fluorescence microscopy, and combinations thereof; and
a plurality of channels arranged in the plate base, each channel of the plurality of channels spaced apart from one or more sides of the plurality of sides, each channel of the plurality of channels having a predetermined depth from the upper surface of the plate base, and each channel of the plurality of channels further having a length and a width, wherein the length is greater than the width;
wherein the plurality of channels are arranged as a plurality of straight or curved branches of a central channel of the plurality of channels, the central channel having a first width or diameter and the plurality of branching channels having widths or diameters which are successively or incrementally smaller than the first width or diameter.

21. A system for magnetically induced rotary translational movement of magnetic particles in a tissue culture plate or tray, the system comprising:
a magnetic device to generate a rotating magnetic field having a time-varying amplitude and polarity; and
a tissue culture plate or tray spaced-apart from the magnetic device a predetermined lateral distance and elevation, the tissue culture plate or tray comprising:
a plate base having an upper surface and a plurality of sides, the plate base having a form factor of a truncated cuboid;
a plurality of channels arranged in the plate base, each channel of the plurality of channels spaced apart from one or more sides of the plurality of sides, each channel of the plurality of channels having a predetermined depth from the upper surface of the plate base, each channel of the plurality of channels further having a length and a width, wherein the length is greater than the width.

22. The system of claim 21, wherein the predetermined lateral distance and elevation is selected from the group consisting of: a pull position laterally offset from the magnet in a first direction; a push position laterally offset from the magnet in a second direction, the second direction opposite the first direction; a centered position having a predetermined elevation above or below the magnet without a lateral offset; an offset position having a predetermined elevation above the magnet with a lateral offset; and combinations thereof.

23. The system of claim 21, further comprising:
an adjustable plate mount removably couplable to the tissue culture plate or tray, the adjustable plate mount configured to position the tissue culture plate or tray at user-selectable, varying distances and elevations from the magnet.

24. The system of claim 21, further comprising:
a heating unit configured to heat contents of the tissue culture plate or tray to a predetermined temperature or to maintain the tissue culture plate or tray at the predetermined temperature.
